(19) United States
(12) Patent Application Publication
(10) Pub. No.: US 2003/0012703 A1
(43) Pub. Date: Jan. 16, 2003

(54) Air cleaning device with optic catalyzer

(76) Inventor: Yuan Huan Lee, Hsin Chu Hsien (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 09/903,551

(22) Filed: Jul. 13, 2001

(51) Int. Cl. A61L 9/20; B01J 19/12

(52) U.S. Cl. 422/121; 422/24; 422/186.3

(57) ABSTRACT

An air cleaning device with an optic catalyzer lamp tube includes a machine body, an optic catalyzer lamp tube and an enforcing convection device. The fan of the enforcing convection device in the interior of the machine body absorbs air from air inlets. When air flows through the optic catalyzer lamp tube, the dust, and germs in the air can be removed so as to achieve the effect of dust and germ-proof. The enforcing convection device serves to transfer cleaning air from the optic catalyzer lamp tube so that the air has the effect of filtering. Therefore, the indoor air may flow convectionally. The dust or germ in the air can be removed gradually so as to improve the quality of air.

### FIELD OF THE INVENTION

The present invention relates to an air cleaning device with an optic catalyzer, and particularly to an air cleaning device with effects of dust-proof, sterilization, air filtering, and cleaning so as to improve the quality of indoor air.

### BACKGROUND OF THE INVENTION

The prior art air cleaning device is utilized to filter and absorb dusts and other floating particles in air. The design of an air cleaning device is based on many factors, such as the extent of air necessary to be removed, volume, and quality of wastes, conditions of air and airflow, etc. These factors affect the air cleaning device in different conditions.

Referring to FIG. 1, a prior art air cleaning device with a filter 11a is illustrated. The filter 11a is formed by various materials, for example, a microporous material made of fabrics, etc., for filtering dusts.

However, the prior air cleaning device has only functions of removing dusts and particles, while functions of such as sterilization and deodorization are not equipped.

### SUMMARY OF THE INVENTION

Accordingly, the primary object of the present invention is to provide an air cleaning device with optic catalyzer, wherein a plurality of optic catalyzer lamp tubes and an enforcing convection device are installed in a machine body, so that outer air may flow through an optic catalyzer lamp tube to achieve the object of dust-proof, sterilization, air filtering, and cleaning to improve the quality of indoor air.

An air-cleaning device with an optic catalyzer having an optic catalyzer lamp tube includes a machine body, an optic catalyzer lamp tube and an enforcing convection device. The fan of the enforcing convection device in the interior of the machine body absorbs air from air inlets. When outer air flows through the optic catalyzer lamp tube, the dust, and germs in the air can be removed so as to achieve the effect of dust and germ-proof. The enforcing convection device serves to transfer cleaning air from the optic catalyzer lamp tube so that the air has the effect of filtering. Therefore, the indoor air may flow convectionally. The dusts or germs in the air can be removed gradually to improve the quality of air.

The various objects and advantages of the present invention will be more readily understood from the following detailed description when read in conjunction with the appended drawing.

### BRIEF DESCRIPTION OF THE DRAWINGS

FIG. 1 is an exploded perspective view of a prior art air cleaning device.

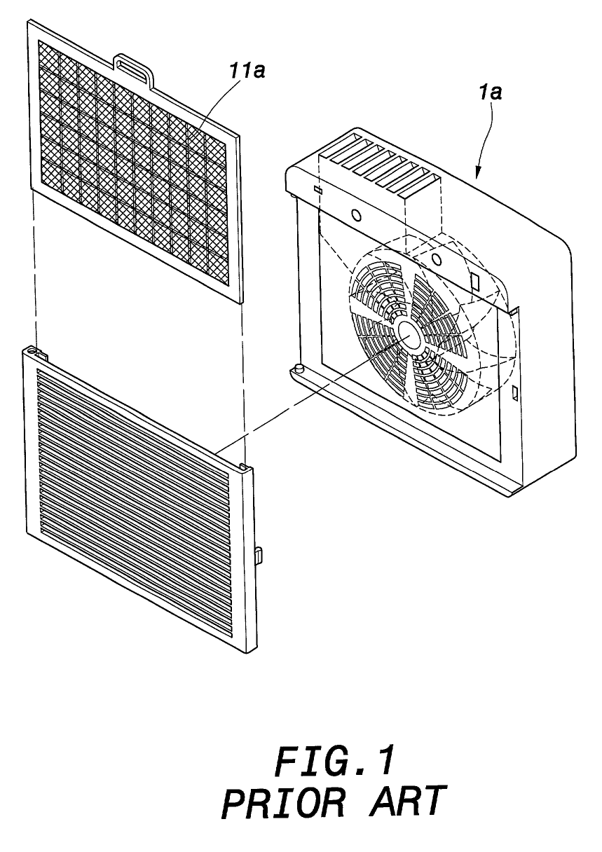

FIG. 2 is an exploded perspective view of the present invention.

FIG. 3 is a schematic perspective view of the present invention.

FIG. 4 is a perspective view of the present invention.

FIG. 5 is a lateral schematic view of the present invention.

### DETAILED DESCRIPTION OF THE PREFERRED EMBODIMENTS

In order that those skilled in the art can further understand the present invention, a description will be described in the following in details. However, these descriptions and the appended drawings are only used to cause those skilled in the art to understand the objects, features, and characteristics of the present invention, but not to be used to confine the scope and spirit of the present invention defined in the appended claims.

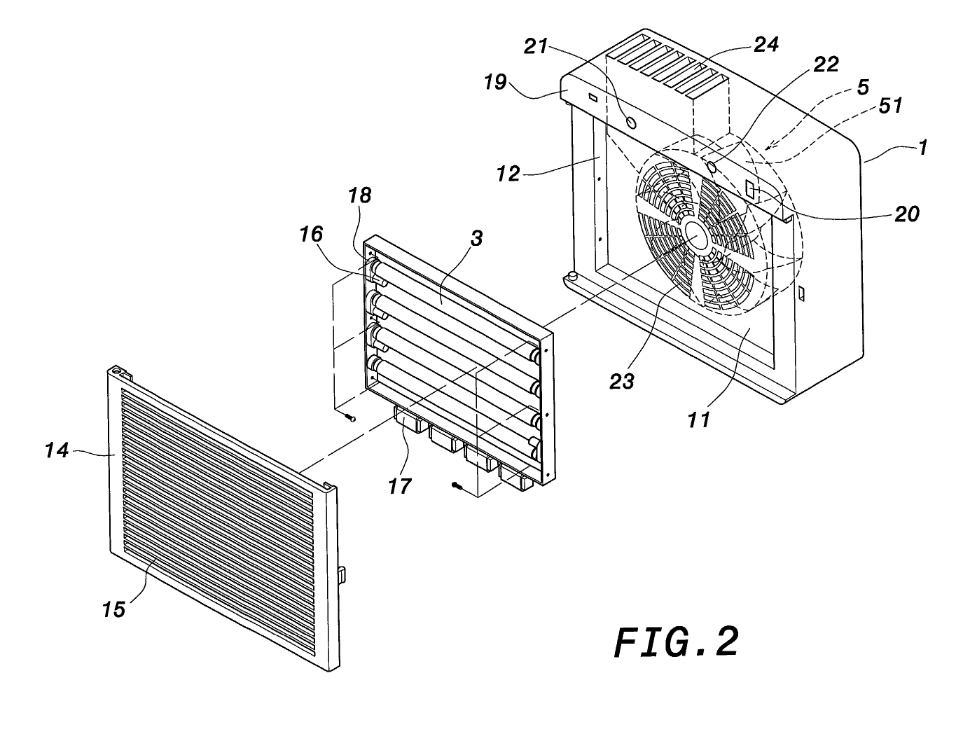

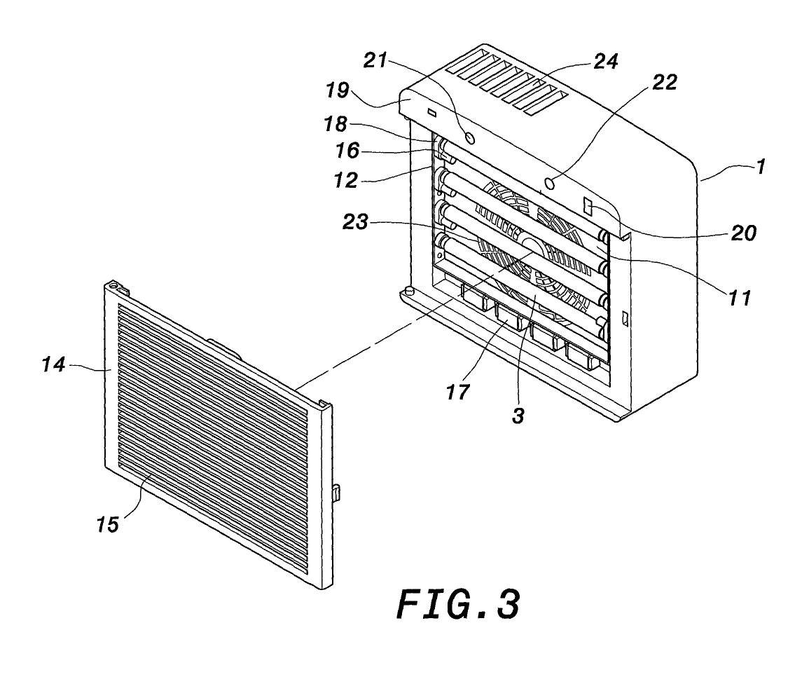

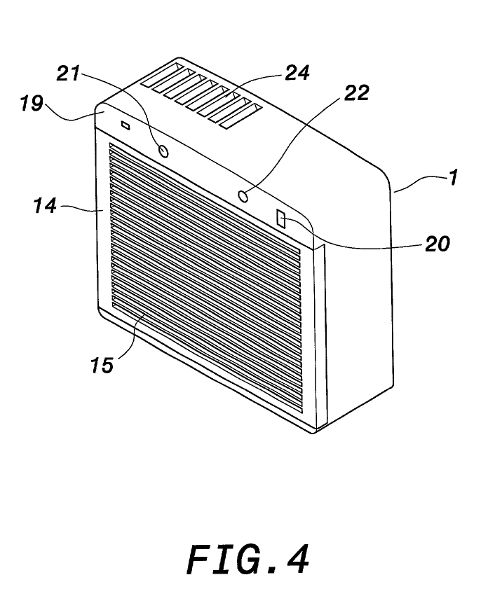

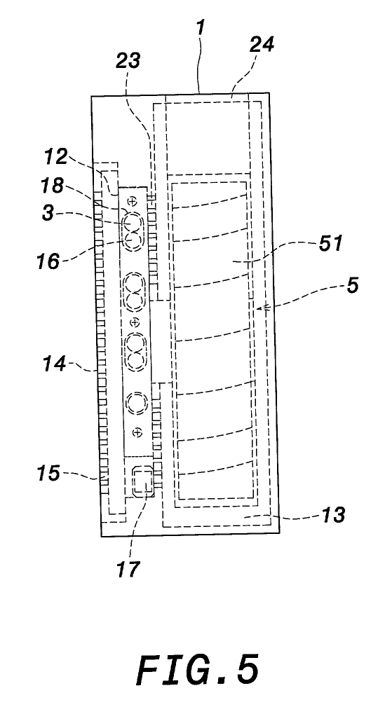

Referring to FIGS. 2 to 5, an air cleaning device with an optic catalyzer of the present invention is illustrated. The air cleaning device includes a machine body 1, one or more optic catalyzer lamp tubes 3, and an enforcing convection device 5. The machine body 1 is a hollow casing. The interior thereof has a spacer 11. The interior of the machine body 1 is spaced as a first receiving space 12 and a second receiving space 13. The second receiving space 13 is positioned behind the first receiving space 12.

The front side of the first receiving space 12 is opened and is communicated with outer environment. A grid plate 14 is buckled thereto. The grid plate 14 is formed with a plurality of air inlets 15. The interior of the first receiving space 12 is installed with a starter 16, a ballast 17, a lamp receptacle 18, and other power circuits for supplying power to the optic catalyzer lamp tubes 3.

The front side of the machine body 1 is installed with a control panel 19 at a top thereof. The control panel 19 is installed with a switch key 20, a wind speed adjusting key 21, and a timing adjuster 22. The switch key 20 is used to control the enforcing convection device 5 and the optic catalyzer lamp tube 3. The wind adjuster 21 is used to control the volume of the speed of the enforcing convection device 5. For example the wind speed can be controlled in one of a strong, a middle, and a weak level. The timing adjusting key 22 can be used to control the timing, for example, timing of 1, 2, 4 or 8 hours.

The spacer 11 is installed with a plurality of vents 23, thereby, air flowing conveniently between the first receiving space 12 and second receiving space 13. A top of the machine body 1 is installed with a plurality of air outlets 24. The air outlets 24 are formed above the second receiving space 13.

The optic catalyzer lamp tubes 3 are installed in the interior of the first receiving space 12. The two ends of the optic catalyzer lamp tube 3 are inserted into the two respective lamp tube receptacles 18 for supplying power to the optic catalyzer lamp tubes 3.

The optic catalyzer lamp tube 3 has a structure like that in the prior art (referring to Taiwan Patent No. 356224). The optic catalyzer lamp tube 3 is formed by an ultraviolet lamp tube, glass fiber cloth, and optic catalyzer films (not shown). The ultraviolet lamp tube is enclosed by the glass fiber of a proper thickness. The glass fiber is a glass fiber sleeve or a glass fiber material. A layer of titanium dioxide optic catalyzer film encloses the glass fiber so as to form an optic catalyzer lamp tube 3.

The enforcing convection device 5 is installed within the second receiving space 13 of the machine body 1 and is formed by a fan 51. The fan 51 is fixed to the spacer 11. When the fan 51 is driven by a proper power. Outer air is drawn into the first receiving space 12 from the air inlets 15. Then, the air passes through the vents 23. The air in the first receiving space 12 is drawn from the air outlets 24 at the top of the machine body 1 so that air may flow convectionally. Therefore, the air-cleaning device with an optic catalyzer of the present invention is formed by abovesaid components.

When outer air flow through the optic catalyzer lamp tube 3, since the ultraviolet lamp tube lights up, the optic catalyzer film oxidizes and decomposes harmful materials (organic or inorganic) in the air to generate water, carbon dioxide, etc. The optic catalyzer may generate a catalyzer reaction under the catalysis of ultraviolet light so that the environmental oxygen and water molecular generates free radicals with a strong oxidization function which decompose almost all materials. The free radicals will destroy a cell membrane of a germ so as to sterilize. Furthermore, it has a preferred deodorization effect and organic decomposition effect. As a result, most waste air can be decomposed, thereby, the present invention having a function of cleaning air.

In the present invention, the fan 51 of the enforcing convection device 5 in the interior of the machine body 1 absorbs air from the air inlets 15. When outer air flows through the optic catalyzer lamp tube 3, the dust, and germs in the air can be removed so as to achieve the effect of dust and germ-proof. The enforcing convection device 5 serves to transfer cleaning air from the optic catalyzer lamp tube 3 so that the air has the effect of filtering. Therefore, the indoor air may flow convectionally. The dusts or germs in the air can be removed gradually to improve the quality of air.

Although the present invention has been described with reference to the preferred embodiments, it will be understood that the invention is not limited to the details described thereof. Various substitutions and modifications have been suggested in the foregoing description, and others will occur to those of ordinary skill in the art. Therefore, all such substitutions and modifications are intended to be embraced within the scope of the invention as defined in the appended claims.

What is claimed is:

1. An air cleaning device with an optic catalyzer comprising:
a machine body having a first receiving space and a second receiving space in an interior thereof; the machine body installed with air inlets and air outlets;
at least one optic catalyzer lamp tube installed in the first receiving space; and
an enforcing convection device installed in the second receiving space;
whereby, when the enforcing convection device is driven, outer air flows into the first receiving space from the air inlets of the machine body; the outer air flows through the optic catalyzer lamp tube and then is exhausted from the air outlets.

2. The air cleaning device with optic catalyzer as claimed in claim 1, wherein a spacer is installed within the machine body; the spacer spaces an inner space of the machine body into the first receiving space and the second receiving space; the second receiving space is at a rear side of the first receiving space; and the spacer is installed with vents.

3. The air cleaning device with optic catalyzer as claimed in claim 1, wherein a front side of the first receiving space of the machine body is opened and is communicated with outer environment; a grid plate is adhered to the first side and has the air inlets.

4. The air cleaning device with optic catalyzer as claimed in claim 1, wherein the air outlets of the machine body is installed at a top of the machine body; and the air outlets are positioned above the second receiving space.

5. The air cleaning device with optic catalyzer as claimed in claim 1, wherein a power supply circuit is installed in the first receiving space of the machine body for supply power to the optic catalyzer lamp tube.

6. The air cleaning device with optic catalyzer as claimed in claim 1, wherein the machine body is installed with a control panel; the control panel is installed with a switch key, a wind speed adjusting key, and a timing adjusting key.

7. The air cleaning device with optic catalyzer as claimed in claim 1, wherein the enforcing convection device is formed by a fan.
